(19) United States
(12) Patent Application Publication
(10) Pub. No.: US 2006/0111447 A1
(43) Pub. Date: May 25, 2006

(54) Inhibiting progressive cognitive impairment

(76) Inventor: Giulio Maria Pasinetti, New York, NY (US)

Correspondence Address: BAKER & BOTTS, 30 ROCKEFELLER PLAZA, NEW YORK, NY, 10112, US

(21) Appl. No.: 11/257,287

(22) Filed: Oct. 24, 2005

Related U.S. Application Data
(63) Continuation of application No. 11/019,408, filed on Dec. 22, 2004, now abandoned.
Application No. 11/019,408 is a continuation of application No. PCT/US03/18402, filed on Jun. 11, 2003.
(60) Provisional application No. 60/388,452, filed on Jun. 13, 2002.

(51) Int. Cl. A61K 31/18

(52) U.S. Cl. 514/602

(57) ABSTRACT

The present invention provides compositions and methods for inhibiting progressive cognitive impairment in persons at risk of developing dementia, for example patients suffering from MCI. It is based, at least in part, on the discovery that nimesulide, at extremely low concentrations, has a number of anti-amyloid effects, including decreasing the level of Aβ₄₀, inhibiting the activity of secretase-γ, and favoring the formation of sAPPα. As extracellular β-amyloid accumulation is associated with pathologic cognitive deterioration, nimesulide may be used to intervene in amyloid plaque formation and thereby retard cognitive decline.

### CROSS-REFERENCE TO RELATED APPLICATION(S)

This application claims priority from U.S. Application Ser. No. 60/388,452 filed Jun. 13, 2002, the entire disclosure of which is incorporated herein by reference.

### 1. INTRODUCTION

The present invention relates to methods and compositions for inhibiting progressive cognitive dysfunction. In particular embodiments, the present invention provides for preventing or slowing the progression of the neurological disorder, Mild Cognitive Impairment.

### 2. BACKGROUND OF THE INVENTION

### 2.1 Mild Cognitive Impairment

While slight memory loss is a normal part of the aging process, a more severe form of memory deficit, termed “Mild Cognitive Impairment” (“MCI”) has been identified which carries a substantial risk of progressing to Alzheimer's Disease. The clinical definition of MCI is somewhat heterogeneous, with “MCI-amnestic” defined as an isolated disorder of memory and “MCI-other” defined as deficits in two or more areas of cognition greater than 1.5 standard deviations below the mean, corrected for age and education. In a recent Report of the Quality Standards Subcommittee of the American Academy of Neurology, the former definition was favored, in that patients with MCI were characterized as suffering from memory impairment, but not demented (Petersen et al., 2001, Neurology 56:1133-1142; “the AAN Report”). The AAN Report estimates that between 6 and 25 percent of MCI patients will progress to Alzheimer's Disease (“AD”) or another form of dementia each year.

In view of this risk, the AAN Report stresses the importance of identifying patients suffering from MCI for closer monitoring. Albert et al. (2001, J. Intl. Neuropsycholog. Soc. (“JINS”) 7:631-639) identified four particular neuropsychological tests which provide assessments of memory and executive function and were found to have predictive value in determining which MCI patients were more likely to develop AD.

The neuropathology of MCI is the subject of active research. Jack et al. (1999, Neurology 52(7):1397-1403) report a correlation between the size of the hippocampus (a structure in the brain associated with memory formation), determined by MRI, and progression of MCI to AD. Morris and Price (2001, J. Mol. Neurosci. 17(2):101-118) report that “widespread amyloid plaques in the neocortex best distinguishes very early stage AD, including MCI stage, and preclinical stages, from healthy brain aging.” Mitchell et al. (2002, Ann. Neurol. 51(2):182-189) report regional pathology in the distribution of phosphorylated tau protein which occurs prior to dementia and correlates with impairment of episodic memory.

### 2.2 Inflammation and Alzheimer's Disease

There is an association between inflammation and Alzheimer's disease, but it is unknown whether inflammation causes the neuropathology or vice-versa. Recent reports indicate that cyclooxygenase-2 (“COX-2”), a proinflammatory enzyme responsible for converting arachidonic acid to prostaglandin E₂, is up-regulated in the brains of AD patients (U.S. Pat. No. 5,985,930 by Pasinetti et al.; International Patent Application No. PCT/US97/21484, Publication No. WO 98/22104, by Pasinetti et al.) and increases as cognitive impairment declines (Ho et al., 2001, Arch. Neurol. 58:487-492). Other classical markers of inflammation such as cytokine expression and HLA-DR immunoreactive microglia are also apparent in AD, but occur in later, more severe phases of the disease (Hull et al., 2002, Curr. Med. Chem. 9(1):83-88). Elevation in the levels of caspases, proteins associated with the cell cycle and apoptosis, have also been reported, but the significance of this, as cause or effect, is unclear (Rohn et al., 2001, Neurobiol. Dis. 8(6):1006-1016; Roth et al., 2001, J. Neuropath. Exp. Neurol. 60(9):829-838).

Epidemiologic studies reviewing the occurrence of AD in arthritis patients, typically long-term users of anti-inflammatory agents, suggest that anti-inflammatory drugs may delay the onset and possibly the progression of AD (McGeer et al., 1996, Neurology 47:425-434). Weggen et al. (2001, Nature 414:159-160) recently reported that some, but not all, NSAIDs lowered amyloidogenic Aβ₄₂ (see section 2.3, below) in cell culture and animal models. However, when anti-inflammatory agents have been administered in a therapeutic context to AD patients, the results have not been consistently positive. Whereas several non-steroidal anti-inflammatory drugs (“NSAIDs”; Rogers et al., 1993, Neurology 43(8):1609-1611) have shown a trend toward disease modification, steroids (quintessential anti-inflammatory agents) did not produce a beneficial effect (Hull et al., 2002, Curr. Med. Chem. 9(1):83-88), obscuring the relationship between anti-inflammatory treatment and symptomatic stabilization or improvement.

Various NSAID agents are still under review for their ability to treat AD. One such agent, the selective COX-2 inhibitor nimesulide, has been found to have a neuroprotective effect against β-amyloid peptide-induced cell death and glutamate toxicity (U.S. Pat. No. 5,985,930 by Pasinetti et al.; International Patent Application No. PCT/US97/21484, Publication No. WO 98/22104, by Pasinetti et al.). In a twelve-week clinical trial, nimesulide was well tolerated in AD patients; no cognitive benefit was observed but this was perhaps due to the short term of the study (Aisen et al., 2002, Neurology 58(7):1050-1054). In a clinical study to assess long term treatment, nimesulide was well tolerated for up to 104 weeks.

### 2.3 Amyloid Plaque Formation

Amyloid plaques (also referred to as “neuritic plaques”), along with neurofibrillary tangles, are neuropathologic features that are substantially more numerous in brain tissue of patients suffering from either MCI or AD than in normal elderly patients. The fundamental unit of the plaques is β-amyloid peptide, a toxic by-product originating in Amyloid Precursor Protein (APP).

APP is a receptor-like type-1 integral transmembrane glycoprotein which may be involved in neuronal growth and synaptic plasticity (Heuvel et al., 1999, Exp. Neurol. 159(2): 441-450) and/or apoptotic cell death (Mbebi et al., 2001, Rev. Neurol. (Paris) 157 Suppl. 10:48). At least three proteases, α, β and γ secretases, are believed to cleave APP (Selkoe, 1996, J. Bio. Chem. 271:18295-18298). FIG. 4 illustrates the relevant products of APP processing. The α-secretase cleaves APP between Lys687 and Leu688, releasing a large soluble protein termed “sAPPα” (Selkoe, 1996, J. Biol. Chem. 271:18295-18298; Sinha and Lieberburg, 1999, Proc. Natl. Acad. Sci. U.S.A. 96:11049-11053 ; Esler and Wolfe, 2001, Science 293:1449). In contrast, the β-secretase (Sinha et al., 1999, Nature 402:537-540) cleaves APP between Met671 and Asp672, producing soluble “sAPPβ”. The remaining 12 kDa C-terminal fragment can then be further cleaved by γ-secretase at either Val711 or Ile713, releasing either amyloid-β peptide “Aβ₄₀” or “Aβ₄₂”, respectively. Because cleavage by α-secretase does not provide a suitable substrate for γ-secretase, increases in α-secretase activity, associated with a decrease in amyloid β peptide formation (Haass and Selkoe, 1993, Cell 75:1039-1042 ), are considered to be anti-amyloidogenic. Conversely, inhibition of amyloid β peptide-generating γ-secretase is being explored as a strategy for treating Alzheimer's disease (Wolfe, 2002, Curr. Top. Med. Chem. 2:371-383).

Reports indicate that whereas the ratio of Aβ₄₀ to Aβ₄₂ secreted from cells is estimated to be 90:10, Aβ₄₂ is the major plaque component (Roher et al., 1993, Proc. Nat. Acad. Sci. U.S.A. 90:10836-10840; Iwatsubo et al., 1994, Neuron 13:45-53; Dovey et al., 1993, Neuroreport 4:1039-1042; Asami-Odaka et al., 1995, Biochemistry 34:10272-10278). It has been suggested that Aβ₄₂ may play a key role in plaque formation, particularly early in the disease process (Rockenstein et al., 2001, J. Neurosci. Res. 66:573-582; Sinha et al., 2000, Ann. N.Y. Acad. Sci. 920:206-208), perhaps by generating Aβ fibrils more readily than Aβ₄₀ through a nucleation mechanism (Jarrett et al., 1993, Biochemistry 32:4693-4697).

Although substantial evidence implicates Aβ₄₂, rather than Aβ₄₀, as an important agent in amyloid plaque formation, there is nevertheless a substantial amount of Aβ₄₀ and amino truncated forms of APP ending at residue 40 in senile plaques in the typical late onset AD brain. Moreover, in several studies Aβ₄₀ appears to be the most abundant peptide in cerebrovascular amyloid deposits (Golde et al., 2000, Biochem. Biophys. Acta 1502:172-187), and has been suggested to play a more significant role than Aβ₄₂ in cerebrovascular aspects of AD (Niwa et al., 2000, J. Cereb. Blood Flow. Metab. 20(12):1659-1668; Niwa et al., 2000, Proc. Natl. Acad. Sci. U.S.A. 97:9735-9740).

Several mutations of APP have been identified in which persons carrying the mutations are at risk for developing AD, corroborating the relationship between APP processing and cognitive decline. In the “Swedish mutation” of APP, an Asn residue is substituted for Lys670 and a Leu residue is substituted for Met671. This results in increased β-mediated cleavage of APP, increases in both Aβ₄₂ and Aβ₄₀, and early onset of AD (Mullan et al., 1992, Nature Gen. 1:345-347; Citron et al., 1992, Nature 360:672-674). The “London mutation” is a substitution of Ile for Val717, which causes an increase in amyloid β peptide formation (presumably due to an increase in γ-secretase activity) associated with familial early-onset AD (Goate et al., 1991, Nature 349:704-706; Suzuki et al., 1994, Science 264:1336-1340). Mutations in APP have facilitated the development of model systems for neurodegenerative dementia.

### 3. SUMMARY OF THE INVENTION

The present invention provides compositions and methods for inhibiting progressive cognitive impairment in persons at risk of developing dementia, for example patients suffering from MCI. It is based, at least in part, on the discovery that nimesulide, at extremely low concentrations, has a number of anti-amyloid effects, including decreasing the levels of Aβ₄₀ and Aβ₄₂, inhibiting the activity of secretase-γ, and favoring the formation of sAPPα. As extracellular β-amyloid accumulation is associated with pathologic cognitive deterioration, nimesulide may be used to intervene in amyloid plaque formation and thereby retard cognitive decline. Without being bound by any particular theory, it is believed that nimesulide may inhibit amyloidogenesis by both COX-dependent as well as COX-independent mechanisms.

### 4. BRIEF DESCRIPTION OF THE FIGURES

FIG. 1. Clinical Dementia Rating Scale developed by Washington University (http://www.adrc.wustl.edu/adrc/cdrScale.html; Morris, 1993, Neurology 43:2412-2414).

FIG. 2A-C. Relative percentage of serum levels of nimesulide reaching the brain parenchyma following chronic treatment with nimesulide of B6SJ1 mice. (A) 2 month old mice were treated with nimesulide in the diet for three months. (B) PGE₂ level was measured in the same brain used to quantitate nimesulide levels. (C) Relative percent of serum levels of nimesulide reaching the brain under defined conditions.

FIG. 3. Serum concentration in humans following chronic treatment with 100 mg nimesulide administered orally twice a day (daily dosage=200 mg).

FIG. 4. Schematic diagram of β-amyloid peptide, sAPPα and sAPPβ production by secretases.

FIG. 5A-D. Levels of soluble APPα and total soluble APP (sAPPα+sAPPβ). The inserts are photographs of Western blots showing immunoreactivity with either monoclonal antibody 6E10 (for sAPPα) or monoclonal antibody 22C11 (for total soluble APP (“sAPP”)). (A) sAPPα levels in cells treated with 1 μM or 5 μM nimesulide; (B) total sAPP levels in cells treated with 1 μM or 5 μM nimesulide; (C) sAPPα levels in cells treated with 1 μM or 5 μM Rofecoxib; and (D) total sAPP levels in cells treated with 1 μM or 5 μM Rofecoxib.

FIG. 6A-C. (A) Nimesulide inhibits γ-secretase activity in CHO cells transfected with the Swedish mutation of APP. (B) Nimesulide inhibits the formation of Aβ₄₂ in the transfected CHO cells of (A). (C) Nimesulide inhibits the formation of Aβ₄₀ in the transfected CHO cells of (A).

FIG. 7A-C. Analysis of (A) 6E10 immunopositive Aβ plaque load and (B) Congo Red birefringent positive Aβ plaque load, in the cingulate cortex, and (C) numerical Congo Red plaque density through the entire cortex, of 16 and 24 month old APPswe/PS1-A246E/hCOX-2 mice, compared to APPswe/PS1-A246E littermates. In (A) and (B), the results are expressed as percentages of the control group and are shown as mean±S.E.M. In (C), the total number of plaques through the entire cortex is shown as the mean±SEM.

FIG. 8A-D. Representative low-power magnification of 6E10 immunopositive Aβ amyloid plaques, showing cingulate cortex of (A) an APPswe/PS1-A246E doubly transgenic mouse and (B) an APPswe/PS1-A246E/hCOX-2 triply transgenic mouse. Low power magnification of Congo-Red positive Aβ plaques in the cerebral cortex of (C) an APPswe/PS1-A246E doubly transgenic mouse and (C) an APPswe/PS1-A246E/hCOX-2 triply transgenic mouse, where the plaques display birefringence under polarized light. Scale bars: A, B 100 μm; C, D 30 μm.

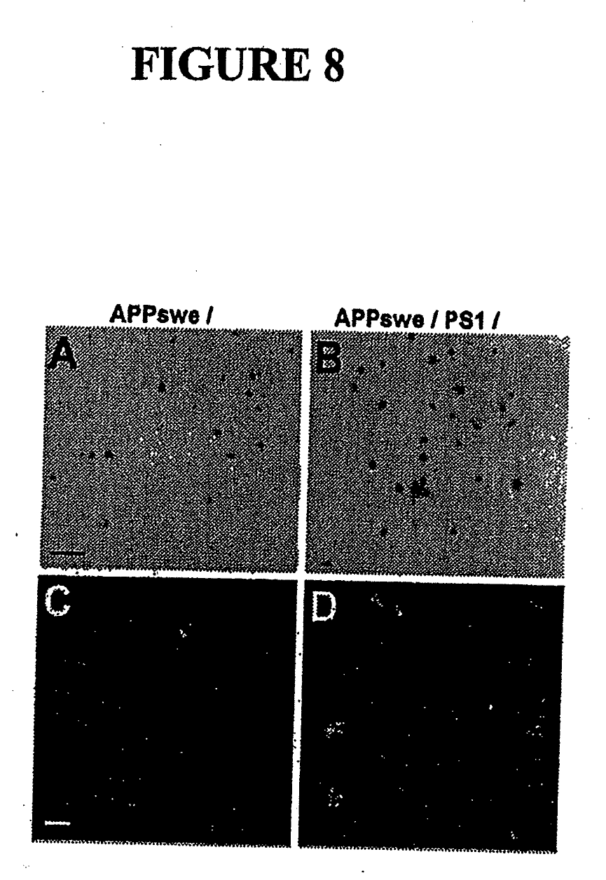

FIG. 9A-D. (A) Total Aβ₄₀ content and (B) total Aβ₄₂ in the cerebral cortex of APPswe/PS1-A246E and APPswe/PS1-A246E/hCOX-2 mice at 16 or 24 months of age. (C) Cortical APP content in 24 month old APPswe/PS1-A246E or APPswe/PS1-A246E/hCOX-2 mice, as determined by (D) Western blot analysis with 22C11 anti-APP antibody. The results are shown as mean±S.E.M.

FIG. 10A-C. (A) Levels of Aβ₄₀ in CHO cells transfected with APPswe and LacZ (open bars) or with APPswe and hCOX-2 (filled bars), which were untreated (control, “0”) or treated with various concentrations of nimesulide. (B) MTT assay of cells used in (A) to demonstrate equal viability in compared cultures. (C) Aβ₄₂ levels in CHO cells transfected with APPswe and LacZ (open bars) or with APPswe and hCOX-2 (filled bars). Data are expressed as means±S.E.M. (percent of control LacZ transfection from three independent studies).

FIG. 11A-B. CHO cells were transfected with APPswe and LacZ (open bars) or with APPswe and hCOX-2(filled bars), as in FIG. 10A-C. (A) γ-secretase activity in cells treated with dimethylsulfoxide (“DMSO”) or nimesulide at concentrations of 10⁻⁹ or 10⁻⁶ molar. (B) Concentration (pg/ml) of Aβ₄₂ in cells treated with dimethylsulfoxide (“DMSO”) or nimesulide at concentrations of 10⁻⁹ or 10⁻⁶ molar.

### 5. DETAILED DESCRIPTION OF THE INVENTION

The present invention provides for methods of inhibiting progressive cognitive impairment by administering effective doses of nimesulide over a treatment period.

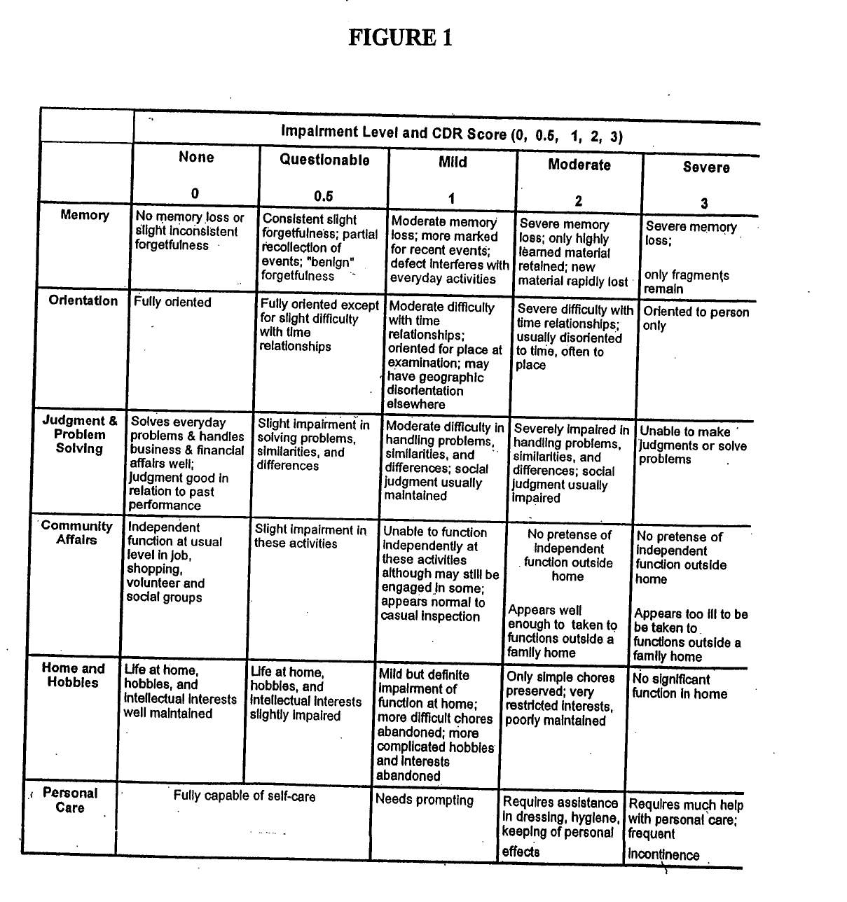

“Inhibiting progressive cognitive impairment” is defined as retarding deterioration in cognition in a subject at risk for developing a progressive cognitive disorder. A deterioration in cognition means a decrease in the ability of a subject to perform mental tasks. There are numerous standardized tests for assessing cognition and identifying deterioration in cognition. For example, but not by way of limitation, cognitive impairment may be measured using the Clinical Dementia Rating Scale (“CDRS”), as set forth in FIG. 1. Other non-limiting examples of such tests include the Mini-Mental Status Exam (“MMSE”; Tangalos, et al., 1996, 71:829-837), Kokmen Short Test of Mental Status (Kokmen et al., 1991, Arch. Neurol. 48:725-728), 7-Minute Screen (Solomon et al., 1998, Arch. Neurol. 55:349-355), and the Memory Impairment Screen (Buschke et al., 1999, Neurology, 52:231-238), and other tests set forth in Table 6 of Petersen et al., 2001, Neurology 56:1133-1142.

All persons aged 50 or greater are considered to be at risk. Particularly at risk are persons suffering from impairment of memory, and more particularly at risk are persons suffering from MCI. A subject may be considered to be suffering from MCI regardless of whether or not such a formal diagnosis has been made. The definition of MCI used herein embraces both art-recognized definitions: (i) an isolated amnestic disorder, preferably where the subject has a CDRS score of 0.5 or less; and/or (ii) deficits in two or more areas of cognition greater than 1.5 standard deviations below the mean. As a non-limiting example, a person may be identified as suffering from MCI by having the following behavioral characteristics: a memory complaint (preferably corroborated); objective memory impairment; normal general cognitive function; intact activities of daily living; and not demented (Petersen et al., 2001, Neurology 56:1133-1142; Petersen, 1999, Arch. Neurol. 56:303-308). Further, a person may be identified as suffering from MCI by having subjective and/or objective memory deficit and a decrease in hippocampal volume of at least 10 percent relative to a matched control (see Du et al., 2001, J. Neurol. Neurosurg. Psychiatry 71(4): 441-447). Alternatively, a person may be identified as suffering from MCI by having subjective and/or objective memory deficit and a decrease in hippocampal glucose metabolism of 20 percent or greater (deLeon et al., 2001, Proc. Natl. Acad. Sci. U.S.A. 98(19): 10966-10971).

An inhibition of progression means that the treated subject's mental status does not deteriorate as quickly as would be expected, based on the cognitive status of an untreated population having similar clinical features. As used herein, the term “inhibition of progression” does not necessarily mean that no cognitive decline will occur. The present invention may, for example, but not by way of limitation, significantly decrease the rate of progression from MCI to AD per year in a nimesulide treated population of MCI patients relative to an untreated population.

In related embodiments, the present invention provides for methods of decreasing levels of Aβ₄₀ and/or Aβ₄₂ peptide, inhibiting γ secretase, and/or increasing the level of sAPPα, in cells of the nervous system, comprising exposing said cells to an effective concentration of nimesulide. Such methods may be particularly directed toward cells of the nervous system which overproduce amyloid β peptide(s). Technology has recently been developed to identify subjects that overproduce amyloid β peptides by determining serum levels of amyloid (deMattos et al., 2002, Science 295:2264-2267); such methods, adapted to apply to human subjects, may be used to identify subjects who would be more likely to benefit from treatment with nimesulide.

The term “nimesulide”, as used herein, refers to a compound, 4-nitro-2-phenoxymethanesulfonanilide having a structure as set forth in Formula I, below:

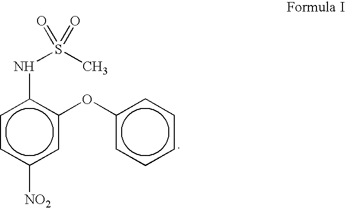

The amount of nimesulide administered may produce a local concentration, in the nervous system, of at least one picomolar, and preferably at least one nanomolar, more preferably at least 0.1 micromolar. The amount of nimesulide administered may produce a serum concentration of at least 10⁻¹¹ molar, preferably at least 10⁻⁸ molar, and more preferably at least one micromolar.

The amount of nimesulide administered per day may be 200 mg per day but preferably is less. The present invention provides, in specific, non-limiting embodiments , for daily dosages of up to 200 mg, between 100 and 200 mg (e.g. 100 mg), between 50 and 100 mg (e.g., 50 mg), or between 10 and 50 mg (e.g. 20 mg) (all ranges are inclusive of their limits). The daily dosage may be administered as a single dose or as divided doses.

The present invention may also be practiced by administering the foregoing daily dosages such that there are days where the patient “skips” treatment—for example, the daily dosage is administered every other day, or every third day, etc.

It is desirable to monitor a subject's liver function tests and stop or suspend treatment if abnormalities arise.

Nimesulide may be administered according to the invention for any desirable duration of time. The “treatment period” is preferably, but not by way of limitation, at least six months.

The present invention accordingly provides for the use of nimesulide for the preparation of a pharmaceutical composition for inhibiting progressive cognitive impairment in persons at risk of developing dementia. In specific, non-limiting embodiments, the present invention provides for the use of nimesulide for the preparation of a pharmaceutical composition for treating MCI. In still other specific, non-limiting embodiments, the present invention provides for the use of nimesulide for the preparation of a pharmaceutical composition for treating a subject suspected of suffering from MCI.

A pharmaceutical composition may be administered by any suitable route, including oral, sublingual, intranasal, intravenous, subcutaneous, rectal, etc., but the preferred route is oral.

A pharmaceutical composition comprising nimesulide may further comprise inactive agents and/or biologically active agents, e.g. one or more vitamins.

### 6. EXAMPLE: NIMESULIDE CROSSES THE BLOOD/BRAIN BARRIER

The tolerability of nimesulide treatment and the ability of the drug to cross the blood/brain barrier was evaluated in mice. Nimesulide was mixed directly with powdered rodent feed in a mixing drum into a homogeneous preparation (1500 mg/kg of diet) and administered for three months. Assuming that each mouse consumed approximately 1 gram of nimesulide-containing feed each day, the daily dosage for each mouse may be calculated to be 1.5 mg nimesulide, equivalent to 50 mg/kg. This dose would be approximately 15 times greater than the recommended anti-inflammatory dose for humans.

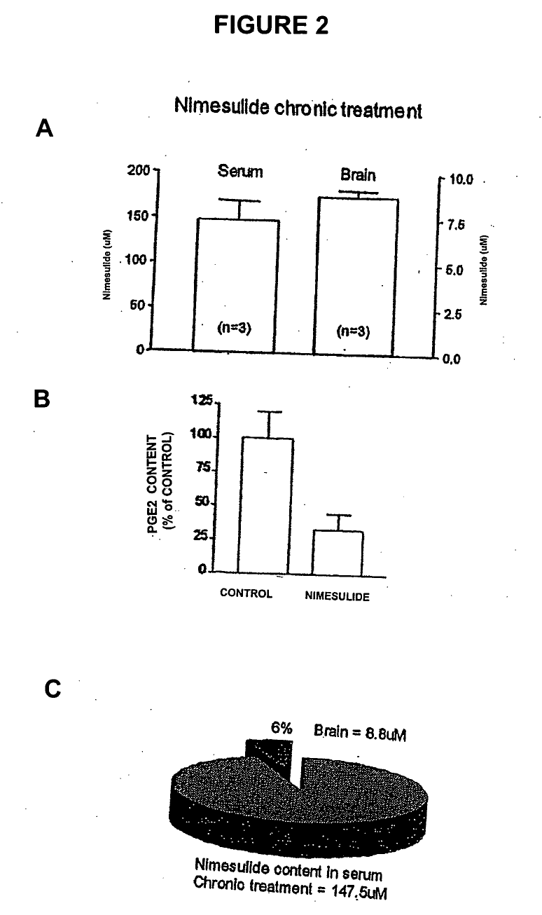

After the 3 month treatment period, the mice were sacrificed and the amounts of nimesulide present in serum and brain were determined. As shown in FIGS. 2A and 2C, the level of nimesulide in the brain was found to be approximately 10 percent of serum levels. When the levels of PGE₂ were measured, nimesulide was determined to have decreased PGE₂ content by approximately 50 percent (FIG. 2B).

At sacrifice, necropsied nimesulide-treated mice did not demonstrate any gross alterations or renal or intestinal abnormalities. In addition, no weight loss was detected in the nimesulide-treated group relative to controls fed unadulterated diet. These observations indicate that nimesulide was well tolerated.

This study confirmed previous evidence that nimesulide can be tolerated even at high pharmaceutical doses and demonstrates that, administered chronically, the drug achieves concentrations of 10-15 percent serum levels in the brain parenchyma.

### 7. NIMESULIDE SERUM LEVELS IN HUMANS

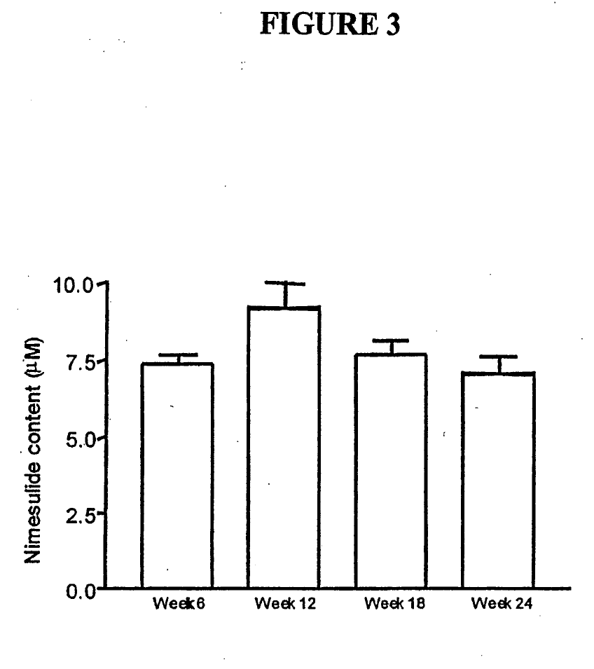

A tolerability study was performed in elderly human subjects (Aisen et al., 2002, Neurology 58(7):1050-1054). Subjects with probable AD were enrolled in a randomized, controlled, parallel group trial designed to assess the tolerability and short-term behavioral effects of nimesulide. In the initial 12-week double-blind phase, subjects were treated with nimesulide 100 mg orally, twice daily (p.o., BID) or matching placebo. During the second 12-week phase all subjects received active drug. Nimesulide serum concentration in subjects that received 100 mg p.o. BID for 24 weeks were assessed. It was found that the serum concentration of nimesulide in subjects undergoing chronic nimesulide treatment with 100 mg p.o. BID for 24 weeks stabilized around 10 micromolar (μM) (FIG. 3). If we assume that approximately ten percent of the serum levels of nimesulide cross the blood/brain barrier in humans (as in mice, see Section 6), then the concentration of nimesulide in the human brain would be calculated to be approximately 1 micromolar.

### 8. NIMESULIDE INCREASES PRODUCTION OF APPα

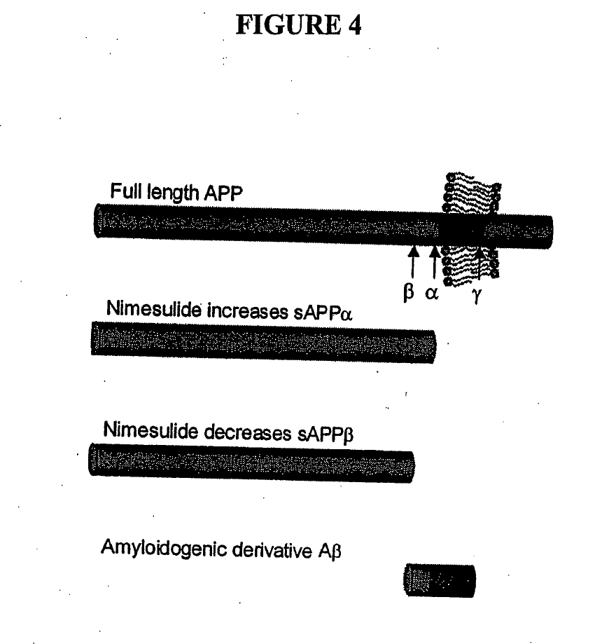

FIG. 4 schematically depicts proteolytic processing of amyloid precursor protein, APP. Cleavage sites for α, β and γ secretases are indicated by arrows. Cleavage by secretase α produces soluble APPα(“sAPPα”). Cleavage with secretase β yields soluble APPβ (“sAPPβ”). Notice that cleavage with secretase β leaves a portion of full length APP in the membrane which, if further cleaved with secretase γ, produces the peptide associated with amyloid plaques, the “β-amyloid peptide.” Favoring production of sAPPα, rather than sAPPβ, should decrease the production of β-amyloid peptide.

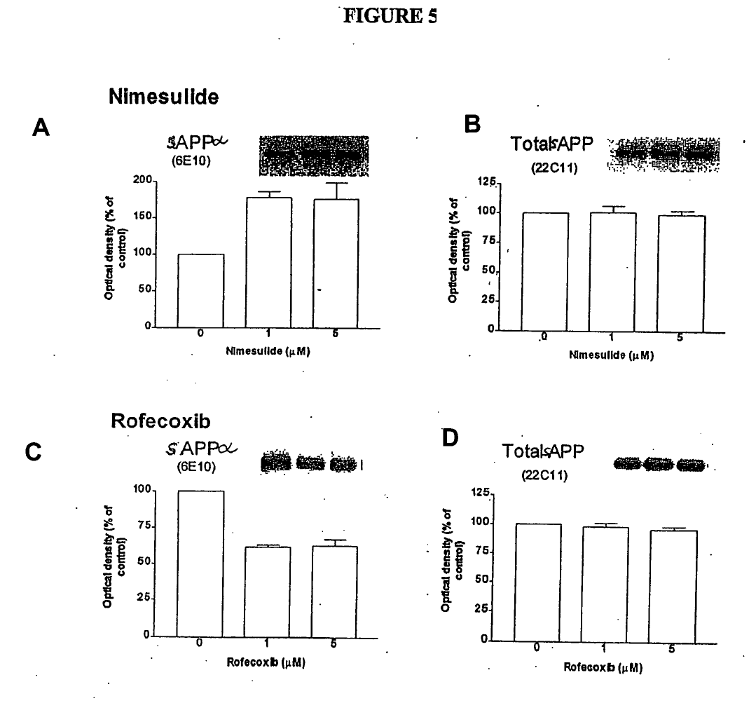

It was observed that exposure of SY5Y neuroblastoma cells in culture to 1 μM and 5 μM concentrations of nimesulide increased levels of sAPPα relative to controls, as indicated by increased immunoreactivity with antibody 6E10 (Senetek, St. Louis, Mo.; FIG. 5A). This change occurred in the absence of an overall elevation in total soluble APP, as demonstrated by immunoreactivity with antibody 22C11 (Chemicon, Temecula Calif.; FIG. 5B). This suggests that nimesulide selectively promoted the formation of sAPPα, while negatively influencing the formation of sAPPβ.

Similar studies were then performed using another selective COX-2 inhibitor, Rofecoxib. Surprisingly, Rofecoxib produced the opposite result of disfavoring sAPPα formation; both 1 μM and 5 μM concentrations decreased 6E10 immunoreactivity (FIG. 5C) relative to total soluble APP (22C11 immunoreactivity, FIG. 5D). This data indicates that Rofecoxib, in this assay, may be pro-amyloidogenic.

### 9. NIMESULIDE INHIBITS γ-SECRETASE AND β-AMYLOID-40 PEPTIDE FORMATION

Chinese Hamster Ovary (“CHO”) cells transfected with nucleic acid encoding the Swedish mutation of APP (which favors amyloid β peptide formation, see Section 2.3; such transfected cells are referred to as “CHO-APPswe” cells) were exposed to varying concentrations of nimesulide for 24 hours and the levels of γ-secretase activity and amyloid β peptides produced were determined.

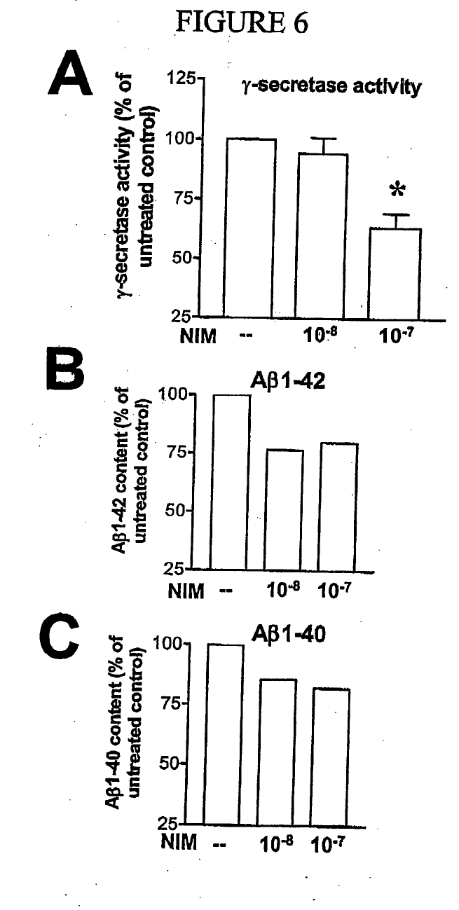

Activity of γ-secretase was measured using a kit from R&D Systems, Inc. (Minneapolis, Minn.; Cat. No. FP003) according to the manufacturer's instructions. Cultured cells were collected in ependorf tubes and lysed by adding Cell Extraction Buffer (1 ml per 25-50×10⁶ cells). Lysates were put on ice for at least 10 minutes and centrifuged at 10,000×g for one minute. The supernatant was transferred into a new tube and kept on ice prior to assay. For the activity assay, 100 μg protein was loaded into each well of a 96-well plate in the presence of γ-secretase specific Aβ38-46 conjugated to reporter molecules EDANS and DABCYL and incubated at 37° C. for 2 hours; cleavage of Aβ38-46 peptide releases a fluorescent signal. Using a fluorescence microplate reader (Perkin Elmer; wavelength: exc. 355 nm; emis. 515 nm) fluorescence was quantified and expressed in arbitrary fluorescence units. As shown in FIG. 6A, nimesulide, at a concentration of 0.1 micromolar, inhibited γ-secretase activity by approximately 40 percent.

The amount of amyloid β peptides produced was measured by an ELISA assay from BioSource (Camarillo, Calif.). Samples were diluted 1:10 in buffer (0.05% (v/v) Tween-20, 1 mM Pefabloc protease inhibitors) followed by centrifugation for 20 minutes at 4° C. Total Aβ₄₀ or Aβ₄₂ were measured by sandwich ELISA following the manufacturer's instructions. Nimesulide, at a concentration of 0.1 micromolar, decreased the amount of Aβ₄₂ and Aβ₄₀ by approximately 20-25 percent (FIGS. 6B and 6C, respectively).

### 10. COX-2 PROMOTES AMYLOID PLAQUE FORMATION IN TRANSGENIC MOUSE MODEL OF AD

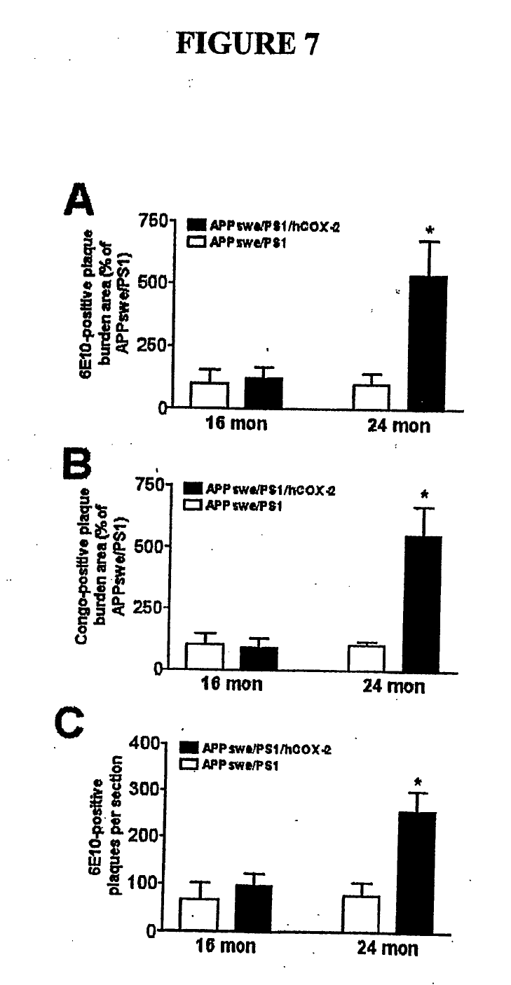

Transgenic mice expressing both the Swedish mutation human amyloid precursor protein mutation (APPswe) and the human presenilin (PS1-A246E) mutation, with resultant AD plaque pathology, were crossed with transgenic mice expressing human (h)COX-2 in neurons. It was reported that at 12 months of age, the APPswe/PS1-A246E/hCOX-2 triple-transgenic mice showed no detectable influence of hCOX-2 on AD neuropathology relative to doubly transgenic APPswe/PS1-A246E mice (Xiang et al., 2002, Neurobiol. Aging 23(3):327-334) although in vitro studies using neurons derived from the transgenic animals showed enhanced amyloid β toxicity in hCOX-2 expressing animals. Subsequent observation of these mice revealed, however, that increased amyloid plaque deposition in triply transgenic mice did occur upon aging. Although no enhancement of plaque pathology was observed at 16 months of age, in 24 month-old triply transgenic mice expressing hCOX-2, increased amyloid plaque deposition was observed relative to doubly transgenic mice lacking the hCOX-2 gene (FIGS. 7A-C and 8A-D). This data is consistent with the results of previous studies showing a relationship between COX-2 expression in amyloidosis and AD (Ho et al., 1999, J. Neurosci. Res. 57(3):295-303; Pasinetti and Aisen, 1998, Neuroscience 87(2):319-324).

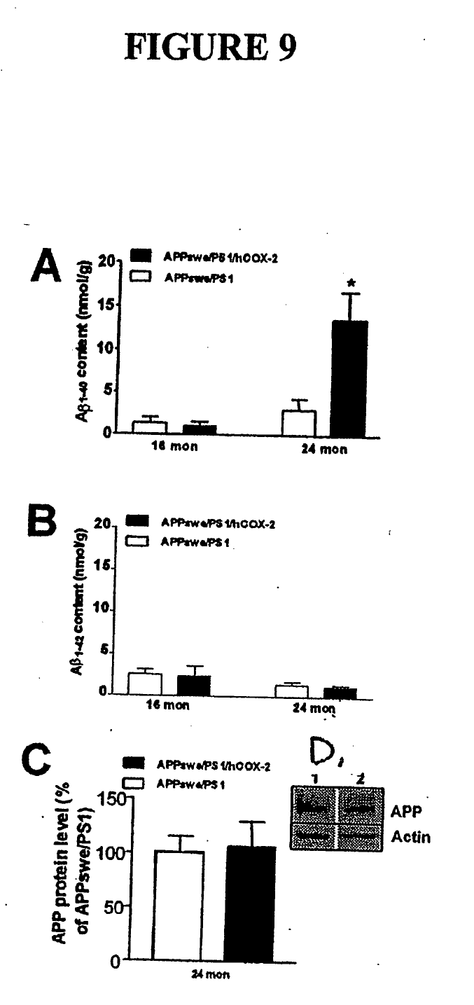

Interestingly, hCOX-2 expression preferentially induced production of Aβ₄₀ rather than Aβ₄₂ (FIG. 9A-D). Aβ₄₀ has been suggested to play a more significant role than Aβ₄₂ in cerebrovascular aspects of AD (Niwa et al., 2000, J. Cereb. Blood Flow. Metab. 20(12):1659-1668; Niwa et al., 2000, Proc. Natl. Acad. Sci. U.S.A. 97:9735-9740). Without being bound by any particular theory, Aβ₄₀ may form fibrils in the presence of a pro-inflammatory facilitating factor. One possible candidate for such a factor is complement ClqB, which is known to promote Aβ aggregation. hCOX-2 has been shown to increase ClqB levels in the brain, and nimesulide has been shown to be able to block that increase (Spielman, 2002, Acta Neuropathol. 103:157-162). Thus, nimesulide may inhibit plaque formation by both decreasing levels of Aβ₄₀ and decreasing levels of ClqB.

### 11. NIMESULIDE INHIBITS COX-2 MEDIATED POTENTIATION OF Aβ40 PRODUCTION

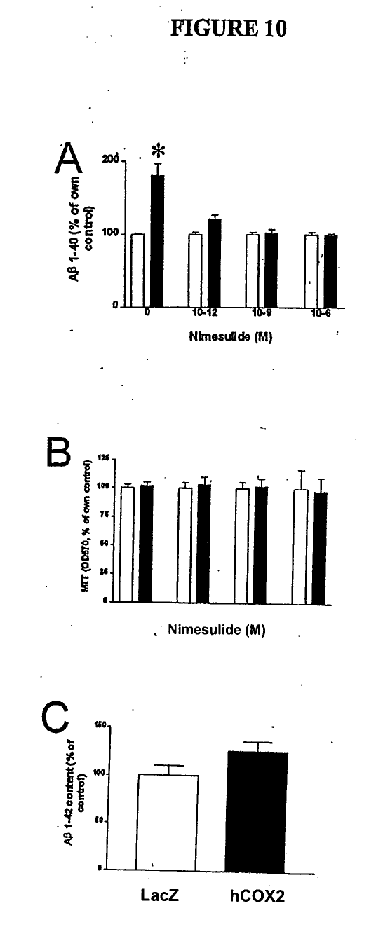

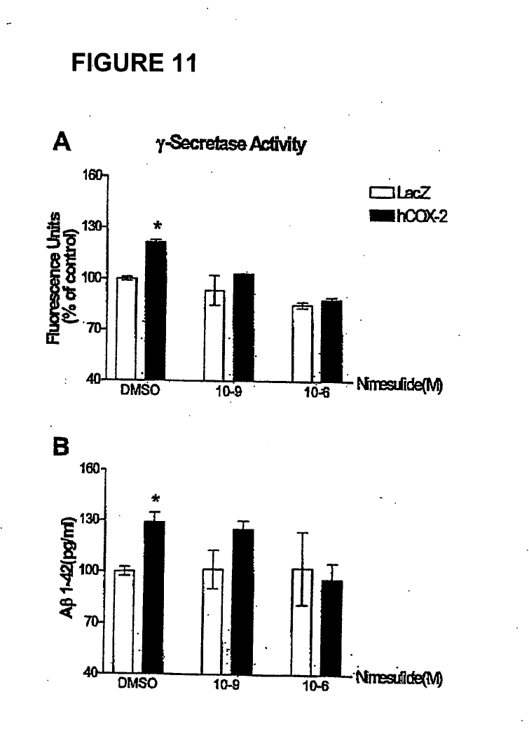

Using an adenovirus vector, hCOX-2 was transfected and expressed in CHO cells which had been stably transfected with APPswe (see above). Consistent with the in vitro studies described in Section 10, 48 hours after transfection, co-expression of hCOX-2 and APPswe resulted in an increase in Aβ₄₀ levels (FIG. 10A). When various concentrations of nimesulide were added to cultures of the doubly transfected CHO cells, this increase in Aβ₄₀ levels could be virtually nullified, even at nimesulide concentrations of 10⁻¹² M (1 picomoloar). In one study, the level of Aβ₄₂ did not appear to be affected by hCOX-2 (FIG. 10C), but see Section 12 and FIG. 11B, infra.

### 12. NIMESULIDE INHIBITS COX-2 MEDIATED POTENTIATION OF Aβ42 PRODUCTION

CHO cells were transfected with APPswe and LacZ (open bars) or with APPswe and hCOX-2 (filled bars), as in the preceding Section 11, and γ-secretase and Aβ₄₂ levels were measured. As shown in FIG. 11A, γ-secretase activity was increased in CHO-APPswe cells transfected with hCOX-2 relative to LacZ control; this increase, of approximately 25 percent in DMSO-treated cells, was decreased to control levels by 10⁻⁹ molar nimesulide, and γ-secretase levels in both hCOX-2 and LacZ-transfected cells were decreased by an additional approximately 10-15 percent by 10⁻⁶ molar nimesulide.

As shown in FIG. 11B, the concentration (pg/ml) of Aβ₄₂ was increased by approximately 30 percent in CHO-APPswe cells transfected with hCOX-2 relative to LacZ control; this level was slightly decreased by 10⁻⁹ molar nimesulide, but was decreased to essentially control levels by 10⁻⁶ molar nimesulide. In contrast to the result of a single study shown in FIG. 10C (which showed no apparent change in Aβ₄₂ levels), the result shown in FIG. 11B arises from multiple independent studies and has been determined to be statistically signifcant.

The conclusions drawn from the experiments described in Sections 11 and 12 are that (1) hCOX-2 preferentially promotes Aβ₄₀ induction, but also promotes Aβ₄₂ induction, albeit to a lesser extent, in CHO-APPswe cells; and (2) treatment of CHO-APPswe cells with nimesulide inhibits hCOX-2 mediated induction of Aβ₄₀ and Aβ₄₂.

Various publications are cited herein, the contents of which are hereby incorporated by reference in their entireties.

I claim:

1. A method of inhibiting progressive cognitive impairment in a subject suffering from Mild Cognitive Impairment comprising administering, to the subject, an effective dose of nimesulide over a treatment period.

2. The method of claim 1, wherein the effective dose of nimesulide results in a serum level of at least 10⁻¹¹ molar in the subject.

3. The method of claim 1, wherein the effective dose of nimesulide results in a serum level of at least 10⁻⁸ molar in the subject.

4. The method of claim 1, wherein the effective dose of nimesulide results in a serum level of at least 10⁻⁶ molar in the subject.
